(19) United States
(12) Patent Application Publication
(10) Pub. No.: US 2025/0380922 A1
(43) Pub. Date: Dec. 18, 2025

(54) APPARATUS AND METHOD FOR QUANTIFICATION OF PULMONARY FUNCTION BASED ON ARTIFICIAL INTELLIGENCE AND MEDICAL IMAGES

(71) Applicant: CORELINE SOFT CO., LTD., Seoul (KR)

(72) Inventors: Ryoungwoo JANG, Seoul (KR); Seungbin BAE, Goyang-si (KR); Donghoon YU, Gimpo-si (KR); Jaeyoun YI, Seoul (KR)

(21) Appl. No.: 19/195,914

(22) Filed: May 1, 2025

(30) Foreign Application Priority Data
May 2, 2024 (KR) 10-2024-0058373
Apr. 29, 2025 (KR) 10-2025-0056647

(51) Int. Cl. A61B 6/00; A61B 6/03; A61B 6/50; G06T 7/00; G06T 7/11; G06T 7/60; G16H 50/20

(52) U.S. Cl. CPC A61B 6/5217; A61B 6/50; G06T 7/0016; G06T 7/11; G06T 7/60; G16H 50/20; A61B 6/032; G06T 2207/10081; G06T 2207/20021; G06T 2207/20084; G06T 2207/20092; G06T 2207/30061

(57) ABSTRACT

A method of quantifying pulmonary function using a medical image includes acquiring or receiving a medical image including anatomical information for a lung region of a patient; segmenting at least one abnormal finding region in the lung region of the medical image using an artificial neural network; and predicting a quantification result related to pulmonary function based on a size of the at least one abnormal finding region.

### CROSS-REFERENCE TO RELATED APPLICATIONS

This application claims priority from Korean Patent Application Nos. 10-2024-0058373 filed on May 2, 2024, and 10-2025-0056647 filed on Apr. 29, 2025, which are incorporated herein by reference in their entireties.

### TECHNICAL FIELD

The present disclosure relates to technology for processing, analyzing, and visualizing medical images, and more particularly, to technology for providing a quantitative evaluation to assist the diagnosis of diseases of the lungs using medical images.

### RELATED ART

The contents described in this section merely provide information about the background art of the present disclosure and do not constitute prior art.

Interstitial lung disease (ILD) is a condition in which changes in the lung parenchyma occur with fibrosis of the lungs. In the past, the disease was diagnosed invasively through lung biopsy, but modern imaging advances have made it possible to diagnose some cases of ILD using imaging alone. However, it is impossible to regenerate damaged lungs to their original state, and the drugs currently on the market do not cure the disease but merely slow down the exacerbation of the disease. Therefore, early diagnosis of the disease is important not only for the quality of life of a patient but also to reduce mortality.

ILD is known to be caused by a combination of environmental factors including smoking and physiological factors including autoimmune diseases. Fortunately, in recent years, there has been an increase in early detection through respiratory screening and chest computed tomography (CT) in comprehensive medical examinations. Accordingly, more patients are actively being examined, and the number of tests is increasing year by year.

To diagnose ILD, a pulmonary function test (PFT) is primarily performed in conjunction with imaging. In performing such a PFT, there are various factors of variation (factors that change data values), and a very large number of instructions are given to control all the factors of variation. Nevertheless, there are many uncontrollable factors, such as patient cooperation and the presence of other medications, which make it difficult to fully trust PFT data. For this reason, image diagnosis and an arterial blood gas test (measurement of oxygen and carbon dioxide tensions) are performed simultaneously with a PFT.

With regard to imaging diagnostics, there has been a technology introduced to automatically diagnose ILD from medical images using artificial intelligence (AI) technology. This is a method of detecting a pattern corresponding to ILD in an image and analyzing the disease from the pattern.

This method is quite useful for detecting ILD but requires a highly sensitive algorithm to detect the disease in its early stages from a medical image. Thus, many disease-like patterns are detected, making it difficult to detect the disease early.

To overcome this limitation, several tests are synthesized. However, it is a complex process for a user to analyze different types of data to draw a comprehensive conclusion, and it is time consuming and costly to train a diagnostician to become proficient. In addition, when limited staff members perform more patient data analysis themselves than in the past, they face the problems of increased fatigue and higher rates of misdiagnosis.

### SUMMARY

A method of evaluating the progression of a lung disease, such as progressive pulmonary fibrosis, is generally based on the presence of at least two of the following: (1) worsening of the patient's symptoms, (2) evidence of worsening in pulmonary function tests (PFTs), and (3) evidence of worsening in radiological exams.

However, evidence of exacerbation from radiological exams is often subjective, depending on the opinion of the medical worker making the diagnosis. Also, even when texture is automatically extracted from medical images, there are no quantified metrics to ensure that a medical worker's judgment corresponds to an automated analysis result.

Accordingly, example embodiments of the present disclosure are provided to substantially obviate one or more problems due to limitations and disadvantages of the related art.

One object of example embodiments of the present disclosure is to detect/segment clinical findings in a medical image and then estimate a pulmonary function test (PFT) indicator to provide a quantified indicator.

Another object of example embodiments of the present disclosure is to quantify evidence of exacerbation from radiological exams and provide the quantified evidence that is comparable with evidence of exacerbation from a PFT, to reduce differences in diagnosing the progression of a lung disease between individual medical workers.

According to an aspect of the present disclosure, there may be provided a method of quantifying pulmonary function using a medical image, the method comprising: acquiring or receiving a medical image including anatomical information for a lung region of a patient; segmenting at least one abnormal finding region in the lung region of the medical image using an artificial neural network; and predicting a quantification result related to pulmonary function based on a size of the at least one abnormal finding region.

The predicting of the quantification result related to the pulmonary function based on the size of the at least one abnormal finding region may comprise: predicting the quantification result related to the pulmonary function by applying a weight predetermined for type of the at least one abnormal finding region to the size of the at least one abnormal finding.

The method of quantifying pulmonary function using a medical image may further comprise: segmenting the lung region of the medical image into a plurality of anatomical regions, wherein each of the at least one abnormal finding region corresponds to any one of the plurality of anatomical regions.

In this case, the predicting of the quantification result related to the pulmonary function based on the size of the at least one abnormal finding region may comprise: predicting the quantification result related to the pulmonary function by applying weights predetermined for abnormal finding regions each corresponding to the plurality of anatomical regions to sizes of the abnormal finding regions each corresponding to the plurality of anatomical regions.

The predicting of the quantification result related to the pulmonary function based on the size of the at least one abnormal finding region may be performed using a linear regression model learning a function of predicting the quantification result related to the pulmonary function by applying a weight predetermined for type of at least one abnormal finding region to the size of the at least one abnormal finding region.

The predicting of the quantification result related to the pulmonary function based on the size of the at least one abnormal finding region may comprise: generating a prediction value of a pulmonary function test (PFT) result as the quantification result.

The predicting of the quantification result related to the pulmonary function based on the size of the at least one abnormal finding region may comprise: predicting a spirometry result, a diffusing capacity, or a lung volume as the quantification result.

The predicting of the quantification result related to the pulmonary function based on the size of the at least one abnormal finding region may comprise: predicting a forced vital capacity (FVC), a forced expiratory volume in one second (FEV1), or a ratio of FEV1 to FVC (FEV1/FVC) as the spirometry result; and predicting a diffusing capacity of the lung for carbon monoxide (DLCO) as the diffusing capacity.

The predicting of the quantification result related to the pulmonary function based on the size of the at least one abnormal finding region comprises: quantifying an effective lung volume based on the size of the at least one abnormal finding region; and generating the prediction value of the PFT result as the quantification result based on the effective lung volume.

In the method of quantifying pulmonary function using a medical image, the at least one abnormal finding region may include an emphysema region, a consolidation region, a ground-glass opacity (GGO) region, a reticulation region, or a honeycomb region.

The method of quantifying pulmonary function using a medical image may further comprise: generating diagnostic assistance information in regard to interstitial lung disease (ILD) or pneumonia of the lung region based on the quantification result related to the pulmonary function.

The method of quantifying pulmonary function using a medical image may further comprise: visualizing the quantification result.

The predicting of the quantification result related to the pulmonary function based on the size of the at least one abnormal finding region may comprise: acquiring a first weight predetermined for type of at least one abnormal finding region; acquiring a second weight by adjusting the first weight in accordance with whether the patient is treated with an antifibrotic; and predicting the quantification result related to the pulmonary function by applying the second weight to the size of the at least one abnormal finding region.

According to an aspect of the present disclosure, there may be provided an apparatus for quantifying pulmonary function using a medical image, the apparatus comprising: a memory configured to store one or more instructions; and a processor configured to load the one or more instructions from the memory and execute the one or more instructions.

In the apparatus according to the present disclosure, the processor, by executing the one or more instructions, may be configured to: acquire or receive a medical image including anatomical information about a patient's lung region; segment at least one abnormal finding region in the lung region of the medical image using an artificial neural network; and predict a quantification result related to pulmonary function based on size of the at least one abnormal finding region.

The processor may be further configured to predict the quantification result related to the pulmonary function by applying a weight predetermined for type of the at least one abnormal finding region to the size of the at least one abnormal finding region.

The processor may be further configured to: segment the lung region of the medical image into a plurality of anatomical regions; match each of the at least one abnormal finding region to any one of the plurality of anatomical regions, and predict the quantification result related to the pulmonary function based on the sizes of the at least one abnormal finding region by applying weights predetermined for abnormal finding regions each corresponding to the plurality of anatomical regions to sizes of the abnormal finding regions each corresponding to the plurality of anatomical regions.

The processor may be further configured to generate a prediction value of a pulmonary function test (PFT) result as the quantification result.

The processor may be further configured to predict a spirometry result, a diffusing capacity, or a lung volume as the quantification result.

The processor may be further configured to: predict a forced vital capacity (FVC), a forced expiratory volume in one second (FEV1), or a ratio of FEV1 to FVC (FEV1/FVC) as the spirometry result; and predict a diffusing capacity of the lung for carbon monoxide (DLCO) as the diffusing capacity.

In the apparatus of quantifying pulmonary function using a medical image, the at least one abnormal finding region may include an emphysema region, a consolidation region, a ground-glass opacity (GGO) region, a reticulation region, or a honeycomb region.

The processor may be further configured to generate diagnostic assistance information about interstitial lung disease (ILD) or pneumonia of the lung region based on the quantification result related to the pulmonary function.

According to an aspect of the present disclosure, there may be provided a method of quantifying pulmonary function using a medical image, the method comprising: acquiring or receiving a medical image including anatomical information for a lung region of a patient; segmenting at least one abnormal finding region in the lung region of the medical image using an artificial neural network; predicting a first quantification result related to pulmonary function based on a size of the at least one abnormal finding region; and visualizing a second quantification result based on a user input in regard to the at least one abnormal finding region.

The method of quantifying pulmonary function using a medical image may further comprise: generating the second quantification result based on the user input in regard to the at least one abnormal finding region.

The generating the second quantification result may comprise: adjusting the at least one abnormal finding region based on the user input in regard to the at least one abnormal finding region; and generating the second quantification result based on the adjusted at least one abnormal finding region.

The visualizing a second quantification result may comprise: visualizing the first quantification result in addition to a first result of segmentation or classification for the at least one abnormal finding region; and visualizing the second quantification result in addition to a second result of segmentation or classification for the adjusted at least one abnormal finding region.

The user input in regard to the at least one abnormal finding region may include: an input to adjust segmented boundaries of the at least one abnormal finding region, an input to change a classification result for the at least one abnormal finding region, an input to perform a segmentation for the at least one abnormal finding region again, and/or an input to perform a classification for the at least one abnormal finding region again.

According to an aspect of the present disclosure, there may be provided an apparatus for quantifying pulmonary function using a medical image, the apparatus comprising: a memory and a processor, by executing one or more instructions loaded from the memory, may be configured to: acquire or receive a medical image including anatomical information about a patient's lung region; segment at least one abnormal finding region in the lung region of the medical image using an artificial neural network; and predict a first quantification result related to pulmonary function based on a size of the at least one abnormal finding region; and visualize a second quantification result based on a user input in regard to the at least one abnormal finding region.

According to an aspect of the present disclosure, there may be provided a method of quantifying pulmonary function using a medical image, the method comprising: acquiring or receiving a medical image including anatomical information for a lung region of a patient; segmenting at least one abnormal finding region in the lung region of the medical image using an artificial neural network; predicting a quantification result related to pulmonary function based on a user input in regard to the at least one abnormal finding region and further based on a size of the at least one abnormal finding region; and visualizing the quantification result.

According to an aspect of the present disclosure, there may be provided an apparatus for quantifying pulmonary function using a medical image, the apparatus comprising: a memory and a processor, by executing one or more instructions loaded from the memory, may be configured to: acquire or receive a medical image including anatomical information about a patient's lung region; segment at least one abnormal finding region in the lung region of the medical image using an artificial neural network; and predict a quantification result related to pulmonary function based on a user input in regard to the at least one abnormal finding region and further based on a size of the at least one abnormal finding region; and visualize the quantification result.

According to an example embodiment of the present disclosure, it may detect/segment clinical findings including six textures in a medical image and then estimate a PFT indicator to provide a quantified indicator.

According to an example embodiment of the present disclosure, it may quantify evidence of exacerbation from radiological exams and provide the quantified evidence that is comparable with evidence of exacerbation from a PFT.

According to an example embodiment of the present disclosure, it may reduce differences in diagnosing the progression of a lung disease between individual medical workers.

Here, the type of finding region, criteria for detecting/segmenting/classifying/determining the finding region, criteria for determining the size of the finding region, and/or the like may be determined in accordance with a specific disease. In addition, a quantification method for calculating the size of a finding region may be selected among clinically known methods.

### BRIEF DESCRIPTION OF THE DRAWINGS

The above and other objects, features and advantages of the present disclosure will be more clearly understood from the following detailed description taken in conjunction with the accompanying drawings, in which:

FIG. 1 is an operational flowchart illustrating a method of quantifying pulmonary function using a medical image according to an example embodiment of the present disclosure;

FIG. 2 is an operational flowchart illustrating a method of quantifying pulmonary function using a medical image according to an alternative embodiment of the present disclosure;

FIG. 3 is an operational flowchart illustrating a part of the process of FIG. 1 in detail in the method according to the example embodiment of the present disclosure;

FIG. 4 is an operational flowchart illustrating another part of the process of FIG. 1 in detail in the method according to the example embodiment of the present disclosure;

FIG. 5 is an operational flowchart illustrating an alternative embodiment of FIG. 4;

FIG. 6 is an operational flowchart illustrating an alternative embodiment of FIG. 1; and

FIG. 7 is a conceptual diagram showing an example of a generalized apparatus and/or a computing system for quantifying the pulmonary function using medical image processing, generating and visualizing diagnostic assistance information for follow-up analysis of a specific disease using a medical image, assisting diagnosis of a specific disease through follow-up according to the embodiments of FIGS. 1 to 6.

### DETAILED DESCRIPTION

Other objects and features of the present disclosure in addition to the above-described objects will be apparent from the following description of embodiments to be given with reference to the accompanying drawings.

The embodiments of the present disclosure will be described in detail below with reference to the accompanying drawings. In the following description, when it is determined that a detailed description of a known component or function may unnecessarily make the gist of the present disclosure obscure, it will be omitted.

Relational terms such as first, second, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed as having formal meanings.

Meanwhile, even when the technology was known before the filing date of the present application, it may be included as part of the configuration of the present disclosure of the present application when necessary, and this will be described herein to the extent that it does not obscure the purpose of the present disclosure. However, in the following description of the configuration of the present disclosure of the present application, detailed descriptions of items that can be clearly understood by those skilled in the art as the technologies known before the filing date of the present application may obscure the purpose of the present disclosure, so that excessively detailed descriptions of known technologies will be omitted.

For example, technologies known before the application of the present disclosure may be used as technology for detecting, segmenting, and classifying specific organs and sub-regions of the human body by processing medical images, technology for generating quantified information by measuring segmented organs or finding regions, and the like, and at least some of these known technologies may be applied as elemental technologies required for practicing the present disclosure. For example, descriptions of elemental technologies required for practicing parts of the configuration of the present disclosure may be replaced by providing notification that the technologies are known to those skilled in the art.

In the prior art literature, lesion candidates are detected using an artificial neural network, and findings are generated by classifying the lesion candidates. Each of the findings includes diagnosis assistance information. The diagnosis assistance information may include quantitative measurements such as the probability that each finding is actually a lesion, the confidence and malignity of the finding, and the sizes and volumes of lesion candidates to which the finding corresponds.

In medical image diagnosis support using an artificial neural network, each finding needs to include probability and quantified confidence as diagnosis assistance information. Since all findings may not be provided to a user, findings are generally filtered by applying a specific threshold, and only findings that are filtered out are provided to the user. In a workflow in which a clinical finding is generated in such a manner that a user who is a radiologist reads a medical image and the results of diagnosis are generated in such a manner that a clinician analyzes the finding, an artificial neural network or automated program may at least partially assist the radiologist in a reading process and a finding generation process and/or the clinician in a diagnosis process.

However, the purpose of the present disclosure is not to claim rights to these known technologies, and the content of the known technologies may be included as part of the present disclosure within the range that does not depart from the spirit of the present disclosure.

Hereinafter, embodiments of the present disclosure will be described in more detail with reference to the accompanying drawings. In order to facilitate overall understanding in the description of the present disclosure, the same reference numerals will be used for the same components throughout the drawings, and redundant descriptions of the same components will be omitted.

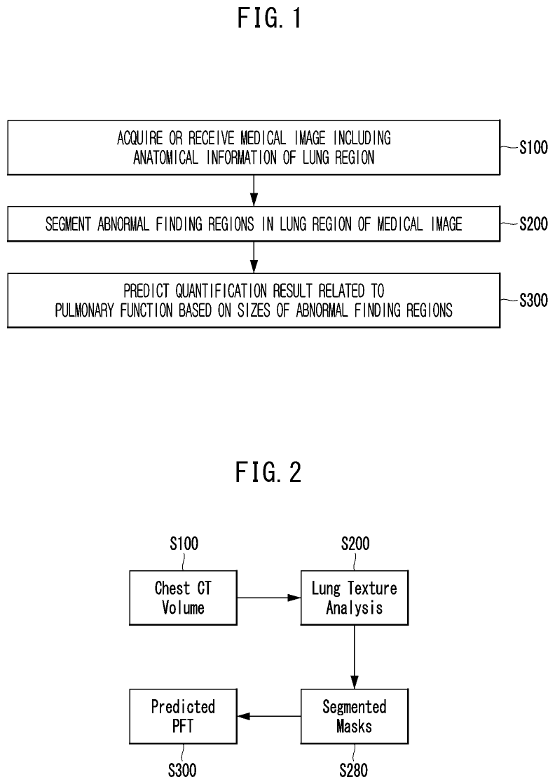

FIG. 1 is an operational flowchart illustrating a method of quantifying pulmonary function using a medical image according to an example embodiment of the present disclosure.

Referring to FIG. 1, the method of quantifying pulmonary function using a medical image according to the example embodiment of the present disclosure may include an operation S100 of acquiring or receiving a medical image including anatomical information about a patient's lung region, an operation S200 of segmenting at least one abnormal finding region in the lung region of the medical image using an artificial neural network, and an operation S300 of predicting a quantification result related to the pulmonary function based on the size(s) of the at least one abnormal finding region.

In the operation S300 of predicting a quantification result related to the pulmonary function based on the size(s) of the at least one abnormal finding region, a quantification result related to the pulmonary function may be predicted by applying weights which are predetermined for types of at least one abnormal finding region to the sizes of the at least one abnormal finding region.

In the method of quantifying pulmonary function using a medical image according to the example embodiment of the present disclosure, the operation S300 of predicting a quantification result related to the pulmonary function based on the sizes of the at least one abnormal finding region may be performed using a linear regression model that has learned a function of predicting the quantification result by applying weights which are predetermined for types of at least one abnormal finding region to the sizes of the at least one abnormal finding region.

In the method of quantifying pulmonary function using a medical image according to the example embodiment of the present disclosure, the at least one abnormal finding region may include an emphysema region, a consolidation region, a ground-glass opacity (GGO) region, a reticulation region, a honeycomb region, or the like. A normal region that is compared with the abnormal finding region may be additionally segmented, and information on the normal region may be provided together with information on the abnormal finding regions.

The method of quantifying pulmonary function using a medical image according to the example embodiment of the present disclosure may further include an operation of generating diagnostic assistance information about interstitial lung disease (ILD) or pneumonia in the lung region based on the quantification result related to the pulmonary function.

A total lung volume is basic information provided with segmentation results of the abnormal finding regions and may be used for calculating a ratio of each of the abnormal finding regions to the total lung volume.

In the operation S300 of predicting a quantification result related to the pulmonary function based on the sizes of the at least one abnormal finding region, a prediction value of a pulmonary function test (PFT) may be generated as the quantification result.

In the operation S300 of predicting a quantification result related to the pulmonary function based on the sizes of the at least one abnormal finding region, a spirometry result, a diffusing capacity, or a lung volume may be predicted as the quantification result.

In the operation S300 of predicting a quantification result related to the pulmonary function based on the sizes of the at least one abnormal finding region, a forced vital capacity (FVC), a forced expiratory volume in one second (FEV1), or a ratio of FEV1 to FVC (FEV1/FVC) may be predicted as the spirometry result.

In the operation S300 of predicting a quantification result related to the pulmonary function based on the sizes of the at least one abnormal finding region, a diffusing capacity of the lung for carbon monoxide (DLCO) may be predicted as the diffusing capacity result.

In the operation S300 of predicting a quantification result related to the pulmonary function based on the sizes of the at least one abnormal finding region, the quantification result related to the pulmonary function based on the sizes of each type of the at least one abnormal finding region.

In this case, the type of the at least one (abnormal) finding region may indicate six texture information described above.

In the operation S300 of predicting a quantification result related to the pulmonary function, by applying the weights predetermined for the type of the at least one abnormal finding region to the sizes of each type of the at least one abnormal finding region.

According to an alternative embodiment of the present disclosure, the operation S300 of predicting a quantification result related to the pulmonary function based on the sizes of the at least one abnormal finding region may include an operation of acquiring first weights predetermined for types of at least one abnormal finding region, an operation of adjusting the first weights in accordance with whether the patient is treated with an antifibrotic to acquire second weights, and an operation of predicting the quantification result related to the pulmonary function by applying the second weights to the sizes of the at least one abnormal finding region.

In other words, the first weights predetermined for the types of abnormal finding regions may be adjusted based on information related to the patient as “additional factors” such that the second weights may be applied. According to the example embodiment, depending on whether the patient is treated with an antifibrotic, the first weights may be applied as the second weights without any change, or changed values of the first weights may be applied as the second weights. Whether the patient is treated with an antifibrotic may be interpreted synonymously with whether drugs are used.

FIG. 2 is an operational flowchart illustrating a method of quantifying pulmonary function using a medical image according to an alternative embodiment of the present disclosure.

Referring to FIG. 2, a chest computed tomography (CT) image may be acquired or received as a medical image (S100). The chest CT image may include three-dimensional anatomical information about a lung region.

An abnormal finding region may be segmented in the lung region using the chest CT image (S200). According to the example embodiment of FIG. 2, this process may be performed as lung texture analysis. Here, lung textures may include a normal region, an emphysema region, a consolidation region, a GGO region, a reticulation region, a honeycomb region, and/or the like. In other words, lung textures may indicate the normal region (normal finding region) and each of the plurality of types of abnormal finding regions.

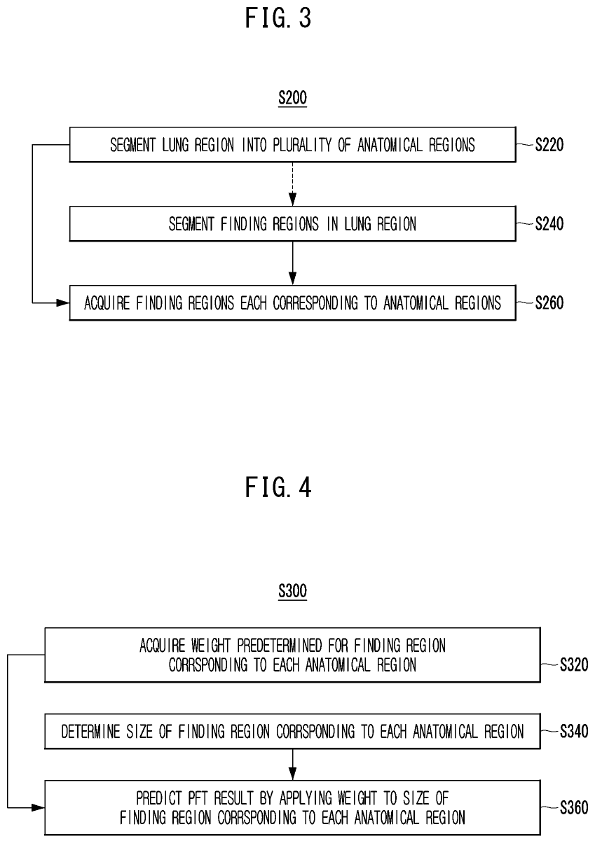

As will be described below with reference to FIGS. 3 and 4, lung texture analysis may be a process of segmenting the lung region into a plurality of anatomical regions and then analyzing and segmenting a lung texture belonging to each anatomical region.

Lung texture analysis may be performed by an artificial neural network (first artificial neural network). The first artificial neural network may be a neural network that has learned the task of classifying and segmenting six lung textures. The first artificial neural network may be a neural network that has learned both a task of segmenting a lung region into a plurality of anatomical regions and a task of classifying and segmenting the six lung textures. Alternatively, a plurality of models may be disposed in the first artificial neural network. Some models may have learned the anatomical region segmentation task, and other models may have learned the task of classifying and segmenting the six lung textures.

After abnormal finding regions are classified into different types, the classification results may be provided as segmented masks (S280). Here, the segmented masks may be provided for the types of abnormal finding regions. According to an alternative embodiment, the segmented masks may be provided for types of abnormal finding regions which have been classified in the plurality of anatomical regions.

For example, when anatomical regions are five lung lobes and finding regions are classified as six types of regions which are a normal region, an emphysema region, a consolidation region, a GGO region, a reticulation region, and a honeycomb region (one normal finding region and five abnormal finding regions), segmented masks may be separately provided depending on 5×6=30 types of anatomical region-finding pairs.

A partial result value of a PFT is predicted for each of the segmented masks, and the partial result values are summed to calculate a PFT value, which may be calculated as a quantification indicator related to the pulmonary function (S300).

FIG. 3 is an operational flowchart illustrating a part (S200) of the process of FIG. 1 in detail in the method according to the example embodiment of the present disclosure.

Referring to FIG. 3, the method of quantifying pulmonary function using a medical image according to the example embodiment of the present disclosure may further include an operation S220 of segmenting the lung region in the medical image into a plurality of anatomical regions and an operation S260 of causing each of the at least one abnormal finding region to correspond to any one of the plurality of anatomical regions.

Here, referring to FIGS. 1 to 3 together, the lung texture analysis operation S200 may include the operation S220 of segmenting the lung region into a plurality of anatomical regions, an operation S240 of segmenting finding regions in the lung region, and an operation S260 of acquiring finding regions that are classified by type to correspond to each of the anatomical regions.

According to the example embodiment of the present disclosure, after the lung region is segmented into the plurality of anatomical regions (S220), finding regions may be segmented for each of the anatomical regions (S240). In the operation S240 of segmenting finding regions, results of the anatomical region segmentation operation S220 may be used.

According to another example embodiment of the present disclosure, the operation S220 of segmenting the lung region into a plurality of anatomical regions and the operation S240 of segmenting the finding regions may be separately and/or independently performed. Here, each finding region may classified as a corresponding anatomical region, and in operation S260, segmentation results of finding regions each mapped (matched or corresponding) (matched or corresponding) to the anatomical regions may be obtained.

The segmentation results of operation S260 may be provided as segmented masks of operation S280.

Here, according to the example embodiment of the present disclosure, anatomical regions may be partial regions of a lung region into which the lung region is segmented.

For example, the anatomical regions may be lung lobes. The right lung may be segmented into 3 lobes, and the left lung may be segmented into 2 lobes.

The 3 lobes of the right lung may be a right upper lobe (RUL), a right middle lobe (RML), and a right lower lobe (RLL), and the 2 lobes of the left lung may be a left upper lobe (LUL) and a left lower lobe (LLL).

According to an alternative embodiment of the present disclosure, the anatomical regions may be segmented into a core region and a rind or peripheral region.

According to another alternative embodiment of the present disclosure, the anatomical regions may be segmented into lung zones. A process of segmenting lung zones may be performed using known technology.

According to the example embodiment of the present disclosure, the operation S220 of segmenting the lung region into a plurality of anatomical regions may be performed independently on different types of anatomical regions. Here, the operation S240 of segmenting the finding regions and the operation S260 of acquiring finding regions each mapped (matched or corresponding) (matched or corresponding) to the anatomical regions may be performed separately on different types of anatomical regions.

For example, operation S220 may be performed on the five lung lobes, and operation S240 and operation S260 may provide information on finding regions each mapped (matched or corresponding) to the five lung lobes. The results may be provided as a first group of segmented masks.

Operation S220 may be performed on the core region and the rind/peripheral regions, and operation S240 and operation S260 may provide information on finding regions each mapped (matched or corresponding) to the core region and the rind/peripheral regions. The results may be provided as a second group of segmented masks.

Operation S220 may be performed on lung-zone regions, and operation S240 and operation S260 may provide information on finding regions each mapped (matched or corresponding) to the lung-zone regions. The results may be provided as a third group of segmented masks.

PFT results may be predicted for each of the first, second, and third groups of segmented masks, and the predicted PFT values may be provided as quantification information.

FIG. 4 is an operational flowchart illustrating another part S300 of the process of FIG. 1 in detail in the method according to the example embodiment of the present disclosure.

Referring to FIG. 4, operation S300 may include an operation S320 of acquiring predetermined weights for types of finding regions each mapped (matched or corresponding) to the anatomical regions.

In operation S300, the size of a finding region mapped (matched or corresponding) to each anatomical region may be determined in accordance with a type of finding region (S340).

Operation S320 and operation S340 may be separately performed. Logically, either one of operation S320 and operation S340 may be performed prior to the other, or both may be performed together (or simultaneously).

Here, the operation S300 of predicting a quantification result related to the pulmonary function based on the sizes of the at least one abnormal finding region may further include an operation S360 of predicting quantification results related to the pulmonary function by applying predetermined weights for types of abnormal finding regions corresponding to each of the plurality of anatomical regions to the sizes of abnormal finding regions corresponding to each of the plurality of anatomical regions.

In the operation S300 of predicting a quantification result related to the pulmonary function based on the sizes of the at least one abnormal finding region in the method of quantifying the pulmonary function using a medical image according to the example embodiment of the present disclosure, operation S360 may be performed using a linear regression model that has learned a function of predicting quantification results related to the pulmonary function by applying weights which are predetermined for types of abnormal finding regions corresponding to each of the plurality of anatomical regions to the sizes of abnormal finding regions corresponding to each of the plurality of anatomical regions.

Referring to the example embodiments of FIGS. 1 to 4 together, according to the quantification method of the present disclosure, lung textures which are finding regions may be analyzed in each of the anatomical regions of the lung.

For example, when the size of a consolidation region of the RUL (consolidation @ right upper lobe) is converted into a PFT prediction value, a weight A1 may be applied, and when the size of a consolidation region of the LUL (consolidation @ left upper lobe) is converted into a PFT prediction value, a weight A2 may be applied.

When the size of a consolidation region of the LUL (consolidation @ left upper lobe) is converted into a PFT prediction value, the weight A2 may be applied, and when the size of an emphysema region of the LUL (emphysema @ left upper lobe) is converted into a PFT prediction value, a weight B2 may be applied.

As a result, different weights may be applied depending on a combination of the 6 texture regions including the normal finding region and the five lobes, and when the sizes of finding regions each mapped (matched or corresponding) to the anatomical regions are converted into PFT prediction values, 6*5=30 or more different weights may be applied.

According to the above embodiment, the anatomical regions may be segmented into not only lobes but also core/rind regions or a combination of lung-zone regions, and thus finding regions may be mapped (matched or corresponding) to each of different anatomical regions and converted into PFT prediction values based on predetermined weights.

According to the example embodiment of the present disclosure, operation S300 may be performed by a rule-based linear regression model that has learned a task of predicting PFT result values for finding regions each mapped (matched or corresponding) to the anatomical regions.

When a new medical image is received (S100), volume information of abnormal findings in each of the anatomical regions may be extracted based on inference results of an artificial neural network that segments abnormal findings in each anatomical region (S200 and S260).

The volume information may be input into a linear regression model.

The linear regression model may output prediction values of PFT results (S300 and S360).

According to another example embodiment of the present disclosure, operation S300 may be performed by an artificial neural network (second artificial neural network) that has learned a regression task of predicting PFT result values for finding regions each mapped (matched or corresponding) to the anatomical regions.

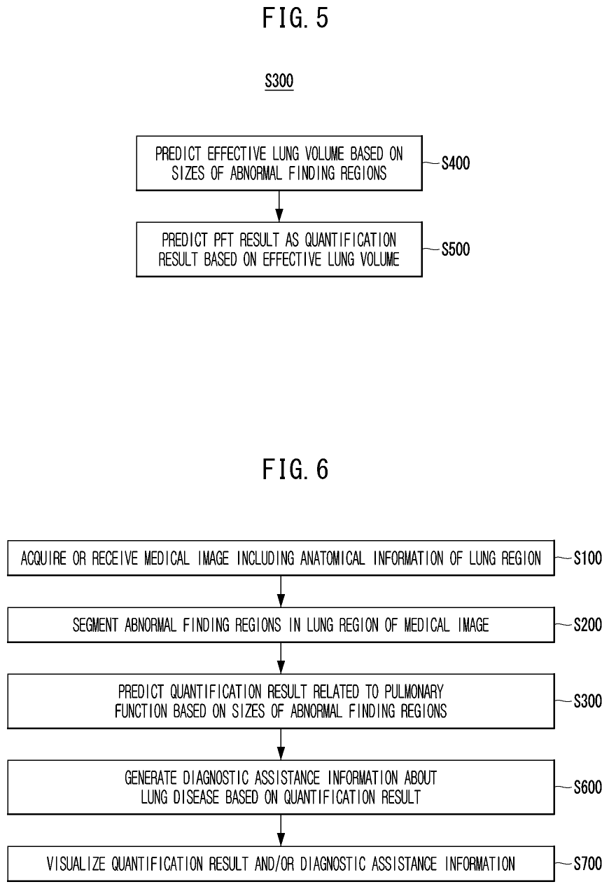

FIG. 5 is an operational flowchart illustrating an alternative embodiment of S300 of FIG. 4.

The operation S300 of predicting a quantification result related to the pulmonary function based on the sizes of the at least one abnormal finding region may include an operation S400 of quantifying an effective lung volume based on the sizes of the at least one abnormal finding region and an operation S500 of generating prediction values of PFT results as quantification results based on the effective lung volume.

The effective lung volume may include input indicators for predicting PFT results. The effective lung volume may be a lung volume that substantially contributes to respiration. In the present disclosure, an effective lung volume may be construed as an estimated/predicted value of a lung volume that substantially contributes to respiration.

Since it is very difficult to directly acquire quantification information of a lung volume that substantially contributes to respiration, an example embodiment of the present disclosure may provide a method of indirectly acquiring quantification information of an effective lung volume that substantially contributes to respiration by predicting PFT results using regression scheme.

According to the above alternative embodiment, in operation S300, PFT results may be predicted by applying the weights predetermined based on types of finding regions corresponding to each of the anatomical regions.

Also, in operation S300, the weights may be adjusted in accordance with the patient's medical/clinical history, and the adjusted weights may be applied.

According to the alternative embodiment of FIG. 5, in operation S400, the effective lung volume may be quantified by applying the predetermined weights based on types of finding regions corresponding to each of the anatomical regions.

In operation S400, the weights may be adjusted based on additional information related to the patient's medical history, and the adjusted weights may be applied.

In operation S500, PFT results may be predicted from the effective lung volume by applying the predetermined weights based on types of finding regions corresponding to each of the anatomical regions.

In operation S500, the weights may be adjusted based on additional information related to the patient's medical history, and the adjusted weights may be applied.

FIG. 6 is an operational flowchart illustrating an alternative embodiment of FIG. 1.

Redundant description of operations S100, S200, and S300 in FIG. 6 which are the same as those in FIG. 1 will be omitted.

Referring to FIG. 6, the method of quantifying the pulmonary function using a medical image according to the example embodiment of the present disclosure may further include an operation S700 of visualizing quantification results generated through operations S100, S200, and S300.

Here, the method of quantifying the pulmonary function using a medical image according to the example embodiment of the present disclosure may further include an operation S600 of generating diagnostic assistance information about a lung disease based on the quantification results. The diagnostic assistance information may include a prediction of a diagnosis of a lung disease based on at least one quantification result.

As described above, the quantification results may include an FVC, an FEV1, FEV1/FVC, and the like and include DLCO and the like. The diagnostic assistance information may be determined based on such a plurality of quantification results or one or more thereof.

When operations S100, S200, and S300 are performed on each of medical images of the same patient obtained at different times, the progression of a lung disease over time may be generated as diagnostic assistance information based on quantification results obtained at different times or opportunities in operation S600.

In general, finding regions may be obtained through thresholding, detection, segmentation, classification, and/or the like in medical images.

A finding region may generally be a lesion or a tumor or may indicate an anatomical structure with a predetermined special shape or structure. Also, a finding region may be an anatomical region that is identified in a medical image through thresholding, detection, segmentation, classification, and/or the like under a special condition.

For example, when an intensity value of a CT image is −950 Hounsfield units (HU), the corresponding region is referred to as a low attenuation area (LAA) and may generally be understood as a finding region corresponding to emphysema. Here, −950 HU is merely a proposed value, and the spirit of the present disclosure is not limited thereto.

As an example of a medical finding region related to a lung disease, a lung parenchyma region may be included. Alternatively, a lung texture region may be included. A lung texture region may be classified as a normal region, a GGO region, a reticular opacity region, a linear opacity region, a nodular opacity region, a honeycomb region, a consolidation region, or the like.

Medical images may be a CT image, a magnetic resonance (MR) image, an X-ray image, and the like, and finding regions may include medical finding regions that may be obtained from a known modality.

Alternative examples of finding regions that may be extracted in consideration of an intensity, a shape, and the like in medical images may include fat, blood, thrombus, and the like.

Finding regions may be obtained through detection, segmentation, or classification based on thresholding results of intensity values.

For example, the distribution of emphysema may be used as an indicator of chronic obstructive pulmonary disease (COPD) symptoms, and aerated lung parenchyma/ventilated functional tissue may generally be considered fibrous tissue that performs the pulmonary function normally.

GGO refers to focal nodular pulmonary infiltrates and may generally be defined as well-delineated nodules in the bronchi or vessels. Diffuse alveolar damage (DAD) and GGO may generally be processed as opacity (OP), and the distribution of OP may be considered an indicator of pneumonia symptoms.

Brightness/intensity value interval or sections may be predetermined for each of fatty tissue (FAT), lymphatic tissue/lymphoedema, leaky/transudate fluids, and exudate fluids, and a small region corresponding to each brightness/intensity value interval or section may be subjected to a thresholding process and visualized.

In addition, a brightness/intensity value interval or section may be set for a lung consolidation region, and the lung consolidation region may be separately subjected to a thresholding process. Lung consolidation is a condition in which the lungs have hardened generally because fluid or cells have replaced air in the alveoli. Lung consolidation may be seen in an X-ray or CT image as a relatively uniform increase in lung OP and little change in lung volume, which may be seen in an air bronchogram. Lung consolidation may also refer to increased OP that prevents identification of pulmonary vessels within a lesion in a CT image.

In the method of quantifying the pulmonary function using a medical image according to the example embodiment of the present disclosure, size information of a finding region may be the volume of the finding region. Here, follow-up information may be a change between the volume of a first finding region obtained from a first medical image at a first time and the volume of a second finding region obtained from a second medical image at a second time.

In the method of quantifying the pulmonary function using a medical image according to the example embodiment of the present disclosure, a non-rigid registration process may be used between the first medical image and the second medical image in order for diagnostic assistance information to include tracking information. Here, a matching process between the first finding region and the second finding region may be additionally included.

The example embodiment of the present disclosure may propose an expression type for generating a change in the size information between finding regions matching each other over time as clinically significant follow-up information. In addition, according to the example embodiment of the present disclosure, a quantitative evaluation means for the progression of a specific disease may be acquired as diagnostic assistance information by quantitatively classifying a combination of quantification results obtained from size information of finding regions matching each other or a change between the quantification results (S600). When the combination of quantification results is classified into a plurality of groups or interval or sections based on one or more thresholds, diagnostic assistance information about the severity of a specific disease may be generated. When the change between the quantification results over time is classified into a plurality of groups or interval or sections based on one or more thresholds, diagnostic assistance information about the progression of a specific disease may be generated.

The example embodiment of the present disclosure may provide a method of visualizing a quantitative evaluation indicator of the severity or progression of a specific disease which is obtained by quantitatively classifying a combination of quantification results obtained from size information of finding regions matching each other over time or a change between the quantification results (S700).

According to the example embodiment of the present disclosure, the diagnostic assistance information based on the quantification results obtained from the finding regions matching each other over time may be presented as an ordered pair such as (a quantification result of the first finding region, a quantification result of the second finding region). According to an alternative embodiment of the present disclosure, a change between quantification results of finding regions matching each other over time may be presented as a difference or ratio between the quantification results.

According to an alternative embodiment of the present disclosure, there may be provided a method of quantifying pulmonary function using a medical image, the method comprising: an operation S100 of acquiring or receiving a medical image including anatomical information for a lung region of a patient; an operation S200 of segmenting at least one abnormal finding region in the lung region of the medical image using an artificial neural network; an alternative operation S300 of predicting a first quantification result related to pulmonary function based on a size of the at least one abnormal finding region; and an alternative operation S700 of visualizing a second quantification result based on a user input in regard to the at least one abnormal finding region.

In this embodiment of the present disclosure, when the user input including request for adjusting and/or changing from user in regard to the at least one finding region, the result adjusted and/or changed based on the user input may be reflected to the second quantification result.

In this embodiment of the present disclosure, in the operation S700, user interface may be provided that provides segmentation and/or classification results of the at least one finding region (including segmentation results of each lung lobe and/or lung sub-area, classification results for each texture, and the like) and PFT quantification results (information) based thereon.

In this embodiment of the present disclosure, in the operation S700, the first quantification result predicted in the operation S300 and the at least one finding region segmented and/or classified in the operation S200 may be visualized.

In this embodiment of the present disclosure, the at least one finding region and the PFT quantification results (information) may be displayed on one screen (or window) or on different screens (or windows).

The method of quantifying pulmonary function using a medical image may further comprise: generating the second quantification result based on the user input in regard to the at least one abnormal finding region while the quantification result predicted in the operation S300 is denoted as the first quantification result.

The generating the second quantification result may comprise: adjusting the at least one abnormal finding region based on the user input in regard to the at least one abnormal finding region; and generating the second quantification result based on the adjusted at least one abnormal finding region.

The visualizing the second quantification result may be performed as a part of the operation S700 described above. The visualizing the second quantification result may comprise: visualizing the first quantification result in addition to a first result of segmentation or classification for the at least one abnormal finding region; and visualizing the second quantification result in addition to a second result of segmentation or classification for the adjusted at least one abnormal finding region.

The user input in regard to the at least one abnormal finding region may include: an input to adjust segmented boundaries of the at least one abnormal finding region, an input to change a classification result for the at least one abnormal finding region, an input to perform a segmentation for the at least one abnormal finding region again, and/or an input to perform a classification for the at least one abnormal finding region again.

In other words, the user's interaction to modify/adjust the finding region may include actions such as adjusting the boundary of the segmented finding region, classifying the previous classification result of the finding region into another texture, and the like. In addition, the user may request that the segmentation and/or classification process of the finding region be performed again using the artificial neural network in order to modify/adjust the finding region.

According to an aspect of the present disclosure, there may be provided a method of quantifying pulmonary function using a medical image, the method comprising: an operation S100 of acquiring or receiving a medical image including anatomical information for a lung region of a patient; an operation S200 of segmenting at least one abnormal finding region in the lung region of the medical image using an artificial neural network; an alternative operation S300 of predicting a quantification result related to pulmonary function based on a user input in regard to the at least one abnormal finding region and further based on a size of the at least one abnormal finding region; and an operation S700 of visualizing the quantification result.

In this embodiment of the present disclosure, when a user modifies/adjusts/changes the AI analysis result (segmentation and/or classification) of the finding region through the user interface, the UI may be provided in which PFT quantitative information is recalculated and displayed in conjunction therewith.

In this case, between the operations S200 and S300, when the user agrees with the segmentation and/or classification results of the abnormal finding region, or when the user directly modifies/changes/adjusts the segmentation and/or classification results of the abnormal finding region via the UI, or when the user requests to re-perform the segmentation and/or classification process of the abnormal finding region using an artificial neural network, the segmentation and/or classification results of the finding region may be either confirmed or modified/adjusted/changed.

After the operation S200, the operation S300 may be performed on the finding region determined through user agreement, modification, or re-segmentation/re-classification.

According to an embodiment of the present disclosure, in the operation S700, a value of the quantification result for individual finding region and/or representative quantification result quantified based on predetermined weights may be visualized using text based on the corresponding value.

According to an embodiment of the present disclosure, in the operation S700, the quantification result for individual finding region and/or representative quantification result quantified based on predetermined weights may be visualized using visual element associated with the individual finding region or anatomical region (structurally segmented sub-region of the lung region, for example, lung lobes, core/peri (rind), and the like) based on the corresponding value of the quantification result (for example, using the color, pattern, design of the finding region or anatomical sub-region).

According to an embodiment of the present disclosure, in the operation S700, the quantification result for individual finding region and/or representative quantification result quantified based on predetermined weights may be visualized using visual element independently visualized from the individual finding region or anatomical region based on the corresponding value of the quantification result (for example, using the size, color, pattern, or design of marker, of icon, and the like). In this case, the correspondence between the independently displayed visual elements and the finding region and/or anatomical region may be expressed using a legend, a label, an annotation, and/or additional visual elements.

Referring to FIGS. 1 to 6, a quantitative distribution of size changes of lung texture finding regions may be acquired and visualized to evaluate the progression of ILD which is also referred to as pulmonary fibrosis, and/or pneumonia. Alternatively, to evaluate the progression of a specific disease, diagnostic assistance information employing follow-up information of PFT quantification results based on the size information of a known finding region may be acquired and visualized.

Although a process of acquiring PFT quantification results to aid in the diagnosis of ILD and/or pneumonia has been mainly described in the above embodiments, the spirit of the present disclosure is not limited to a specific embodiment. It will be clearly understood by those of ordinary skill in the art that the embodiments of the present disclosure are applicable to other lung diseases for which pulmonary function quantification results obtained from the sizes of finding regions in a lung region may be used.

According to an example embodiment of the present disclosure, it may detect/segment clinical findings including six textures in a medical image and then estimate a PFT indicator to provide a quantified indicator.

According to an example embodiment of the present disclosure, it may quantify evidence of exacerbation from radiological exams and provide the quantified evidence that is comparable with evidence of exacerbation from a PFT.

According to an example embodiment of the present disclosure, it may reduce differences in diagnosing the progression of a lung disease between individual medical workers.

Here, the type of finding region, criteria for detecting/segmenting/classifying/determining the finding region, criteria for determining the size of the finding region, and/or the like may be determined in accordance with a specific disease. In addition, a quantification method for calculating the size of a finding region may be selected among clinically known methods.

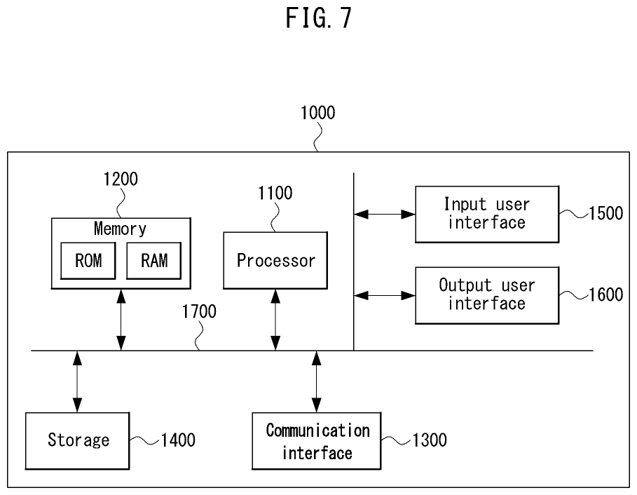

FIG. 7 is a conceptual diagram of an example of a generalized apparatus for and/or computing system for performing medical image processing, analysis and visualization, diagnosis assistance, and quantifying the pulmonary function using medical images, an apparatus for generating and visualizing diagnostic assistance information for follow-up analysis of a specific disease using a medical image, and an apparatus or computing system for supporting the diagnosis of a specific disease through follow-up analysis that may perform at least a part of the processes of FIGS. 1 to 6.

At least a part of process of a method of quantifying the pulmonary function using medical images using medical image processing, analysis, visualization, diagnostic assistance, and the like according to an example embodiment of the present disclosure, a method of generating and visualizing diagnostic assistance information, and/or a method of supporting the diagnosis of a specific disease through follow-up analysis may be performed by a computing system 1000 of FIG. 7.

Referring to FIG. 7, the computing system 1000 according to an embodiment of the present disclosure may include a processor 1100, memory 1200, a communication interface 1300, storage 1400, an input interface 1500, an output interface 1600, and a bus 1700.

The computing system 1000 according to an embodiment of the present disclosure includes at least one processor 1100 and memory 1200 configured to store instructions that instruct the at least one processor 1100 to perform at least one step. At least some steps of the method according to an embodiment of the present disclosure may be performed in such a manner that the at least one processor 1100 loads instructions from the memory 1200 and executes them.

The processor 1100 may refer to a central processing unit (CPU), a graphics processing unit (GPU), or a dedicated processor on which the methods according to embodiments of the present disclosure are performed.

Each of the memory 1200 and the storage 1400 may be formed of at least one of a volatile storage medium and a non-volatile storage medium. For example, the memory 1200 may be formed of at least one of read-only memory (ROM) and random access memory (RAM).

Furthermore, the computing system 1000 may include the communication interface 1300 that performs communication through a wireless network.

Furthermore, the computing system 1000 may further include the storage 1400, the input interface 1500, and the output interface 1600.

Furthermore, the individual components included in the computing system 1000 may be connected by the bus 1700 and communicate with each other.

Examples of the computing system 1000 of the present disclosure may include a desktop computer, a laptop computer, a notebook, a smartphone, a tablet personal computer (PC), a mobile phone, a smart watch, smart glasses, an e-book reader, a portable multimedia player (PMP), a portable game console, a car navigation device, a digital camera, a digital multimedia broadcasting (DMB) player, a digital audio recorder, a digital audio player, a digital video recorder, a digital video player, and a personal digital assistant (PDA) that are capable of communication.

An apparatus for quantifying pulmonary function using a medical image according to an example embodiment of the present disclosure may include: a memory 1200 configured to store one or more instructions; and a processor 1100 configured to load the one or more program instructions from the memory 1200 and execute the one or more program instructions. The processor 1100, by executing the one or more program instructions.

The processor 1100, by executing the one or more program instructions, may acquire or receive a medical image including anatomical information of a patient's lung region, segment at least one abnormal finding region in the lung region of the medical image using an artificial neural network, and predict quantification results related to the pulmonary function based on the size(s) of the at least one abnormal finding region.

The processor 1100 may predict the quantification results related to the pulmonary function by applying at least one weight predetermined for type(s) of the at least one abnormal finding region to the size(s) of the at least one abnormal finding region, for predicting the quantification results related to the pulmonary function based on the size(s) of the at least one abnormal finding region.

The processor 1100, by executing the one or more program instructions, may segment the lung region in the medical image into a plurality of anatomical regions; and match each of the at least one abnormal finding region to any one of the plurality of anatomical regions.

In the case of predicting quantification results related to the pulmonary function based on the sizes of the at least one abnormal finding region, the processor 1100 may predict the quantification results related to the pulmonary function by applying weights predetermined for abnormal finding regions each corresponding to the plurality of anatomical regions to the sizes of the abnormal finding regions each corresponding to the plurality of anatomical regions, for predicting quantification results related to the pulmonary function based on the size(s) of the at least one abnormal finding region.

The processor 1100 may generate prediction values of PFT results as quantification results, for predicting quantification results related to the pulmonary function based on the sizes of the at least one abnormal finding region.

The processor 1100 may predict a spirometry result, a diffusing capacity, or a lung volume as a quantification result, for predicting quantification results related to the pulmonary function based on the sizes of the at least one abnormal finding region.

The processor 1100 may predict an FVC, an FEV1, or a ratio between the FEV and the FEV1 (FEV1/FVC) as the spirometry result, for predicting quantification results related to the pulmonary function based on the sizes of the at least one abnormal finding region.

The processor 1100 may predict a DLCO as the diffusing capacity, for predicting quantification results related to the pulmonary function based on the sizes of the at least one abnormal finding region.

In the apparatus for quantifying the pulmonary function using a medical image according to the example embodiment of the present disclosure, the at least one abnormal finding region may include an emphysema region, a consolidation region, a GGO region, a reticulation region, a honeycomb region, or the like.

The processor 1100, by executing the one or more program instructions, may generate diagnostic assistance information about ILD or pneumonia of the lung region based on the quantification results related to the pulmonary function.

According to an aspect of the present disclosure, there may be provided an apparatus for quantifying pulmonary function using a medical image, the apparatus comprising: a memory 1200 and a processor 1100, by executing one or more instructions loaded from the memory 1200, may be configured to: acquire or receive a medical image including anatomical information about a patient's lung region (S100); segment at least one abnormal finding region in the lung region of the medical image using an artificial neural network (S200); and predict a first quantification result related to pulmonary function based on a size of the at least one abnormal finding region (S300); and visualize a second quantification result based on a user input in regard to the at least one abnormal finding region (S700).

The processor 1100 may generate the second quantification result based on the user input in regard to the at least one abnormal finding region.

The processor 1100 may adjust the at least one abnormal finding region based on the user input in regard to the at least one abnormal finding region; and generate the second quantification result based on the adjusted at least one abnormal finding region, for the generating the second quantification result.

The processor 1100 may visualize the first quantification result in addition to a first result of segmentation or classification for the at least one abnormal finding region; and visualize the second quantification result in addition to a second result of segmentation or classification for the adjusted at least one abnormal finding region, for the visualizing the second quantification result.

In the apparatus for quantifying pulmonary function using a medical image, the user input in regard to the at least one abnormal finding region may include: an input to adjust segmented boundaries of the at least one abnormal finding region, an input to change a classification result for the at least one abnormal finding region, an input to perform a segmentation for the at least one abnormal finding region again, and/or an input to perform a classification for the at least one abnormal finding region again.

According to an aspect of the present disclosure, there may be provided an apparatus for quantifying pulmonary function using a medical image, the apparatus comprising: a memory 1200 and a processor 1100, by executing one or more instructions loaded from the memory, may be configured to: acquire or receive a medical image including anatomical information about a patient's lung region (S100); segment at least one abnormal finding region in the lung region of the medical image using an artificial neural network (S200); and predict a quantification result related to pulmonary function based on a user input in regard to the at least one abnormal finding region and further based on a size of the at least one abnormal finding region (S300); and visualize the quantification result (S700).

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM, or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer, or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A method of quantifying pulmonary function using a medical image, the method comprising:
acquiring or receiving a medical image including anatomical information for a lung region of a patient;
segmenting at least one abnormal finding region in the lung region of the medical image using an artificial neural network; and
predicting a quantification result related to pulmonary function based on a size of the at least one abnormal finding region.

2. The method of claim 1, wherein the predicting of the quantification result related to the pulmonary function based on the size of the at least one abnormal finding region comprises:
predicting the quantification result related to the pulmonary function by applying a weight predetermined for type of the at least one abnormal finding region to the size of the at least one abnormal finding.

3. The method of claim 2, further comprising:
segmenting the lung region of the medical image into a plurality of anatomical regions, wherein each of the at least one abnormal finding region corresponds to any one of the plurality of anatomical regions,
wherein the predicting of the quantification result related to the pulmonary function based on the size of the at least one abnormal finding region comprises:
predicting the quantification result related to the pulmonary function by applying weights predetermined for abnormal finding regions each corresponding to the plurality of anatomical regions to sizes of the abnormal finding regions each corresponding to the plurality of anatomical regions.

4. The method of claim 1, wherein the predicting of the quantification result related to the pulmonary function based on the size of the at least one abnormal finding region is performed using a linear regression model learning a function of predicting the quantification result related to the pulmonary function by applying a weight predetermined for type of at least one abnormal finding region to the size of the at least one abnormal finding region.

5. The method of claim 1, wherein the predicting of the quantification result related to the pulmonary function based on the size of the at least one abnormal finding region comprises:
generating a prediction value of a pulmonary function test (PFT) result as the quantification result.

6. The method of claim 5, wherein the predicting of the quantification result related to the pulmonary function based on the size of the at least one abnormal finding region comprises:
predicting a spirometry result, a diffusing capacity, or a lung volume as the quantification result.

7. The method of claim 6, wherein the predicting of the quantification result related to the pulmonary function based on the size of the at least one abnormal finding region comprises:
predicting a forced vital capacity (FVC), a forced expiratory volume in one second (FEV1), or a ratio of FEV1 to FVC (FEV1/FVC) as the spirometry result; and
predicting a diffusing capacity of the lung for carbon monoxide (DLCO) as the diffusing capacity.

8. The method of claim 7, wherein the predicting of the quantification result related to the pulmonary function based on the size of the at least one abnormal finding region comprises:
quantifying an effective lung volume based on the size of the at least one abnormal finding region; and
generating the prediction value of the PFT result as the quantification result based on the effective lung volume.

9. The method of claim 1, wherein the at least one abnormal finding region includes an emphysema region, a consolidation region, a ground-glass opacity (GGO) region, a reticulation region, or a honeycomb region.

10. The method of claim 9, further comprising:
generating diagnostic assistance information in regard to interstitial lung disease (ILD) or pneumonia of the lung region based on the quantification result related to the pulmonary function.

11. The method of claim 1, further comprising:
visualizing the quantification result,
wherein the predicting of the quantification result related to pulmonary function based on the size of the at least one abnormal finding region comprises:
predicting the quantification result related to pulmonary function based on a user input in regard to the at least one abnormal finding region.

12. The method of claim 1, wherein the predicting of the quantification result related to the pulmonary function based on the size of the at least one abnormal finding region comprises:
acquiring a first weight predetermined for type of at least one abnormal finding region;
acquiring a second weight by adjusting the first weight in accordance with whether the patient is treated with an antifibrotic; and
predicting the quantification result related to the pulmonary function by applying the second weight to the size of the at least one abnormal finding region.

13. A method of quantifying pulmonary function using a medical image, the method comprising:
acquiring or receiving a medical image including anatomical information for a lung region of a patient;
segmenting at least one abnormal finding region in the lung region of the medical image using an artificial neural network;
predicting a first quantification result related to pulmonary function based on a size of the at least one abnormal finding region; and
visualizing a second quantification result based on a user input in regard to the at least one abnormal finding region.

14. An apparatus for quantifying pulmonary function using a medical image, the apparatus comprising:
a memory configured to store at least one instruction; and
a processor configured to execute the at least one instruction, wherein the processor is configured to:
acquire or receive a medical image including anatomical information about a patient's lung region;
segment at least one abnormal finding region in the lung region of the medical image using an artificial neural network; and
predict a quantification result related to pulmonary function based on size of the at least one abnormal finding region.

15. The apparatus of claim 14, wherein, the processor is further configured to predict the quantification result related to the pulmonary function by applying a weight predetermined for type of the at least one abnormal finding region to the size of the at least one abnormal finding region.

16. The apparatus of claim 15, wherein the processor is further configured to:
segment the lung region of the medical image into a plurality of anatomical regions;
match each of the at least one abnormal finding region to any one of the plurality of anatomical regions, and
predict the quantification result related to the pulmonary function based on the sizes of the at least one abnormal finding region by applying weights predetermined for abnormal finding regions each corresponding to the plurality of anatomical regions to sizes of the abnormal finding regions each corresponding to the plurality of anatomical regions.

17. The apparatus of claim 14, wherein, the processor is further configured to generate a prediction value of a pulmonary function test (PFT) result as the quantification result.

18. The apparatus of claim 17, wherein, the processor is further configured to predict a spirometry result, a diffusing capacity, or a lung volume as the quantification result.

19. The apparatus of claim 18, wherein, the processor is further configured to:
predict a forced vital capacity (FVC), a forced expiratory volume in one second (FEV1), or a ratio of FEV1 to FVC (FEV1/FVC) as the spirometry result; and
predict a diffusing capacity of the lung for carbon monoxide (DLCO) as the diffusing capacity.

20. The apparatus of claim 14, wherein the at least one abnormal finding region includes an emphysema region, a consolidation region, a ground-glass opacity (GGO) region, a reticulation region, or a honeycomb region, and
wherein the processor is further configured to generate diagnostic assistance information about interstitial lung disease (ILD) or pneumonia of the lung region based on the quantification result related to the pulmonary function.
